(19) United States
(12) Patent Application Publication
(10) Pub. No.: US 2007/0088333 A1
(43) Pub. Date: Apr. 19, 2007

(54) Method and system for infusing an osmotic solute into a patient and providing feedback control of the infusing rate

(75) Inventors: Howard Levin, Teaneck, NJ (US); Mark Gelfand, New York, NY (US)

Correspondence Address: NIXON & VANDERHYE, PC, 901 NORTH GLEBE ROAD, 11TH FLOOR, ARLINGTON, VA, 22203, US

(73) Assignee: G&L CONSULTING, LLC, New York, NY (US)

(21) Appl. No.: 11/549,182

(22) Filed: Oct. 13, 2006

Related U.S. Application Data
(60) Provisional application No. 60/725,640, filed on Oct. 13, 2005.

(51) Int. Cl. A61K 9/22

(52) U.S. Cl. 604/890.1

(57) ABSTRACT

A patient intravenous (I.V.) infusion pump and biosensors, such as urine volume and sodium concentration sensors, are combined in an infusion system to infuse controlled amount of osmotic agent, such as hypertonic saline, into a blood vessel of a patient. A control subsystem is responsive to the biosensors output and configured to automatically adjust the infusion rate of the infusion pump based on said output. The resulting therapy increases urine output to resolve fluid overload and edema.

### BACKGROUND OF THE INVENTION

This application claims the benefit of U.S. Provisional Patent Application Ser. No. 60/725,640, filed Oct. 13, 2005, the entirety of which is incorporated by reference.

The invention relates to an infusion system that monitors volume and composition of urine and other biofeedback parameters and infuses hypertonic saline or other osmotic agents into the patient's vein based on a programmed control algorithm and the biofeedback parameters. The invention may be applied to treat patients with fluid overload, edema, diuretic resistance and heart failure.

Sodium is an atom, or ion, that carries a single positive charge. The sodium ion may be abbreviated as Na. Sodium can occur as a salt in a crystalline solid. Sodium chloride (NaCl further called salt for simplicity), sodium phosphate (Na2HPO4) and sodium bicarbonate (NaHCO3) are commonly occurring salts. These salts can be dissolved in water. Dissolving in water involves the complete separation of ions, such as sodium and chloride in NaCl. Medical grade pure sterile solution of salt, used for intravascular injections or I.V. infusion, is commonly called saline.

About 40% of the sodium in a human body is contained in bone. Approximately 2-5% of the sodium occurs within organs and cells and the remaining 55% is in blood plasma water and other extracellular (interstitial) fluids. The amount of sodium in blood plasma is typically 140 mM, a much higher amount than is found in intracellular sodium (about 5 mM). This asymmetric distribution of sodium ions is essential for life. It makes possible nerve conduction, the passage of nutrients into cells, and the maintenance of blood pressure.

The body continually regulates its handling of sodium. When dietary sodium is too high or low, the intestines and kidneys respond to adjust concentrations to normal. During the course of a day, the intestines absorb dietary sodium while the kidneys excrete a nearly equal amount of sodium into the urine. If a low sodium diet is consumed, the intestines increase their efficiency of sodium absorption, and the kidneys reduce its release into urine.

The concentration of sodium in the blood plasma depends on two parameters: (A) the total amount of sodium and (B) the amount of water in arteries, veins, and capillaries (the circulatory system). The body uses separate mechanisms to regulate sodium and water, but they work together to correct blood pressure. Too low a concentration of sodium, or hyponatremia, can be corrected by increasing sodium or by decreasing body water (i.e. by free water diuresis, excretion of diluted urine). The existence of separate mechanisms that regulate sodium concentration account for the fact that there are numerous diseases that can cause hyponatremia, including diseases of the heart, kidney, pituitary gland, and hypothalamus.

Fluid overload and edema are a common and serious medical conditions that result from various illnesses. Fluid accumulations in the interstitial space in the lungs or the brain are particularly dangerous and often require intensive care. The most common therapy for fluid overload is oral and I.V. diuretics—drugs that increase urine output of the patient. In most cases diuretics are effective. In some cases patients develop resistance to diuretics and a different or adjunct therapy is needed. One effective therapy of fluid overload is I.V. infusion of an osmotic agent such as, for example, hypertonic saline (NaCl salt-in-water solution). Saline is called hypertonic if its salt content exceeds that of normal blood serum (plasma water). Isotonic or normal saline contains 0.9% NaCl dissolved in water. Half-normal saline contains 0.45% NaCl. Hypertonic saline may contain 1.0 to 7.5% NaCl. Generally, infusion of higher than 2.0% hypertonic saline requires special central vein cannulation, as opposed to a more convenient peripheral I.V. For the purpose of this discussion all crystalloid replacement fluids are called saline, but it is understood that I.V. solutions can contain other additives such as in commonly used Ringer's, Lactated Ringer's, PlasmaLyte, that are all polyionic crystalloid fluids that closely mimic plasma electrolyte concentrations (with or without bicarbonate precursors). A solution of 5% dextrose is an isotonic solution of dextrose in water; the dextrose is rapidly metabolized, thus this essentially results in the administration of free water.

Hypertonic saline I.V. is effective in medical management of cerebral (brain) edema and elevated intracranial pressure (ICP). It is a critical component of perioperative care in neurosurgical practice. Traumatic brain injury (TBI), arterial infarction, venous hypertension/infarction, intracerebral hemorrhage (ICH), subarachnoid hemorrhage (SAH), tumor progression, and postoperative edema can all generate clinical situations in which ICP management is a critical determinant of patient outcomes. Use of hypertonic saline and other osmotic agents is among the most fundamental tools to control ICP. Recently several scientific papers taught the counterintuitive use of hypertonic saline to treat congestive heart failure (CHF or simply heart failure) patients with fluid overload resistive to diuretics. CHF patients retain salt and water to maintain blood pressure and their salt intake is severely limited by the traditional therapy paradigm.

Paterna S, Di Pasquale P, Parrinello G, et al. in “Changes in brain natriuretic peptide levels and bioelectrical impedance measurements after treatment with high-dose furosemide and hypertonic saline solution versus high-dose of furosemide alone in refractory congestive heart failure: a double-blind study” (J Am Coll Cardiol 2005;45:1997-2003; further called Patena Paper) and Stevenson et al. in JACC Vol. 45, No. 12, 2005 Editorial Comment on the Patena Paper describe and comment on results from the randomized study of 94 patients hospitalized with clinical volume overload. The study suggests that the administration of sodium may paradoxically treat the sodium-retaining state. For acute diuresis, very high doses of loop diuretic furosemide (500 to 1,000 mg) were administered twice daily with either hypertonic saline or vehicle infusion concomitantly. Patients receiving hypertonic saline had greater volume loss and were discharged sooner, with better renal function and higher serum sodium.

According to Stevenson, the mechanisms by which in the acute phase of CHF the I.V. infusion of excess saline load facilitated diuresis are open to interpretation and complex. Unmistakably though, there was a larger amount of free water diuresis in the hypertonic saline group. This may relate in part to an acute osmotic effect of hypertonic saline to increase mobilization of extravascular fluid into the central circulation and renal circulation. Direct intratubular effects of sodium flooding may overwhelm the postdiuretic NaCl retention and over time may reduce the diuretic “braking” phenomenon by which fluid escaping past the ascending limb is captured downstream. Neurohormone levels may have been suppressed by hypertonic saline. Both increased intravascular volume and greater delivery of sodium to the distal tubule should inhibit the rennin-angiotensin-aldosterone system. Inhibition of aldosterone release could explain the lower relative potassium excretion in the high sodium group. Reduction in angiotensin II levels could lead also to a decrease in antidiuretic hormone (ADH) vasopressin release despite temporary increase in serum osmolarity. There may also be a small contribution of increased intravascular volume to stimulation of the low-pressure and high-pressure baroreceptors that inhibit vasopressin release. Decreased levels of vasopressin could reduce the aquaporin channels through which water is reabsorbed, leading to the greater free water excretion observed. Reduced vasopressin also might also decrease compensatory over-expression of the sodium transporter in the ascending limb, which diminishes diuretic effect.

Regardless of its mechanisms of action, hypertonic saline therapy could be a useful clinical tool to force diuresis and resolve fluid overload in CHF patients. It is not currently used in routine clinical practice since many concerns are raised in regard to safety and nursing labor involved in the implementation of such therapy. This invention addresses these issues to answer an unmet need for a simple, automated and safe osmotic agent (i.e. hypertonic saline) therapy that could be used in a number of clinical applications such as CHF, brain edema and others to force diuresis, free water excretion, normalize blood plasma sodium concentration or facilitate therapy with diuretics.

### SUMMARY OF THE INVENTION

Applicants realize that fluid retention in some patients results from low sodium content of blood plasma and can be overcome by the I.V. infusion of hypertonic saline. Sodium is a vital electrolyte. Its excess or deficit in blood serum can cause hypernatremia or hyponatremia that can result in abnormal heart rhythm, coma, seizures and death. Administration of an effective therapy with hypertonic saline requires careful monitoring and tight controls. A system and a method have been developed to reduce fluid overload and edema and force diuresis in patients with heart failure and other conditions leading to fluid retention, that do not respond to conventional drug therapy. The system and method provides controlled infusion of an osmotic agent (i.e. hypertonic saline) into the patient's I.V. that is safe and easy to use.

A novel patient infusion, monitoring and control system has been developed that, in one embodiment, comprises:

A. A source of a solution of a blood compatible osmotic I.V. infusible agent such as hypertonic saline,

B. An infusion pump and an I.V. set for controlled delivery of the agent to the patient,

C. A biofeedback sensors connected to the patient that allow monitoring and guiding of the therapy,

D. A microprocessor based controller responsive to the biofeedback signals and is configured to adjust the infusion rate of the pump based on the output of the biofeedback sensors controlling the infusion of the osmotic agent.

In an embodiment that targets therapy of CHF patients, the biofeedback component is comprised of a urine volume monitoring device and a sensor monitoring sodium concentration in urine. The infusion pump is designed for accurate volume delivery. The concentration of sodium in the infusion fluid is known. This allows the controller to calculate the amount of sodium and water delivered to the patient (the “ins”). Urine monitoring measures the amount of water and sodium excreted by the patient (the “outs”). The system balances (the “ins” and “outs”) the total sodium amount in the patient's body water and achieves the desired sodium concentration in plasma. Optionally gradual controlled increase of sodium concentration in serum can be achieved by: a) removal of excess free water in urine, and b) net positive (“ins” over “outs”) addition of small amounts of sodium gradually over hours and days of therapy. As a result, free water excretion is increased, while sodium concentration in blood is maintained within the desired and safe range or increased gradually and safely as desired.

It is understood that the osmotic agent can be a blood compatible small molecule solute other than sodium, such as for example urea. It is preferred that the osmotic agent is normally present in the blood plasma and interstitial water and is excreted by kidneys. It is also understood that the biofeedback may be a physiologic parameter indicative of total or local (in a compartment) body fluid volume such as intracranial pressure (ICP). While the placement of an ICP monitor is invasive, the benefits of ICP monitoring are felt to offset this factor in ICU patients with severe brain trauma. Percutaneous devices (e.g., ventriculostomy catheters) for use in monitoring ICP are commercially available in a variety of styles and from a number of sources. The biofeedback also may be a direct measurement of an osmotic agent and particularly sodium concentration in blood performed using blood chemistry sensors such as, for example, an i-STAT Device manufactured by Abbot Health Care.

In one example, the control system includes a measuring or monitoring sensor as part of or responsive to sodium in the urine collection system and configured to determine the urine output from the patient and a controller responsive to the meter. Typically, the urine collection system includes a urinary catheter connected to the urine collection chamber. In one embodiment, the meter is a weighing mechanism for weighing urine in the collection chamber and outputting a value corresponding to the weight of the urine to the controller. The controller and the weighing mechanism can be separate components or the controller and the weighing mechanism may be integrated. Other types of meters which measure urine output (e.g., volume or flow rate), however, are within the scope of this invention.

Typically, the controller is programmed to determine the rate of change of the urine weight, the rate of change of the urine sodium concentration, to calculate a desired infusion rate based on the rate of change of the urine weight, and to adjust the infusion rate of the infusion pump based on the calculated desired infusion rate to replace sodium lost in urine in a more concentrated solution than urine sodium concentration. As a result net loss of free water is achieved and blood serum sodium concentration is increased, which is the desired goal of the therapy.

It is preferred that the controller subsystem includes a user interface which is configured to allow the user to set a desired serum concentration level achieved in a predetermined time period. The user interface may also include a display indicating the net water and sodium gain or loss, and a display indicating the elapsed time. The user interface can be configured to allow the user to set duration of replacement and to allow the user to set a desired net fluid balance in hourly steps or continuous ramp rate. The control subsystem may also include an alarm subsystem including an air detector. The control subsystem is responsive to the air detector and configured to stop the infusion pump if air exceeding a specified amount is detected. The alarm subsystem may be responsive to the urine collection system and configured to provide an indication when the urine collection system has reached its capacity. The alarm subsystem may also be responsive to the infusion system and configured to provide an indication when the infusion subsystem is low on infusion fluid.

The system may further include a diuretic administration system and/or a blood chemistry sensor responsive to changes of blood sodium concentration. The system may further include a biosensor directly responding to intracranial pressure or the interstitial fluid pressure in a body compartment where edema is present.

A method of removing excess interstitial fluid from the patient with fluid overload and edema in accordance with this invention includes the steps of:

A. Monitoring a biological sensor responsive to a physiologic variable;

B. Controlling the infusion pump based on the said parameter; and

C. Infusing osmotic agent into the patient's blood.

The step of monitoring may comprise measuring the urine output volume and composition. The step of measuring the urine output may further include weighing the urine output by the patient. Typically, the step of adjusting the infusion rate includes determining the rate of excretion of sodium in the urine of the urine output by the patient, calculating a desired infusion rate based on the rate of change of the urine sodium, and adjusting the infusion rate based on the calculated desired infusion rate.

The method may further include the steps of setting a goal (desired or target value) net sodium balance level (net loss or gain) to be achieved by the control algorithm in a predetermined time period, displaying the net fluid and sodium gain or loss, displaying the elapsed time, setting a duration of therapy of the patient, and/or detecting air during the step of infusing the patient with the fluid containing an osmotic agent and automatically stopping infusion if air exceeding a specified amount if detected.

Presumably, as a result, diuresis of a patient is achieved by removal of free water while increasing delivery of sodium to the kidney. Other benefits to the patient, such as vasodilatation, improved heart function, reduced hormone levels and improved kidney function can be expected. Typically, for the proposed method, sodium concentration in urine is substantially lower than in the infused fluid. While the same absolute amount of sodium, thus returned to the patient, may be the same, negative net balance (loss) of water can be achieved. For example, urine Na concentration can be 100 mEq/L and the infusion fluid sodium concentration can be 300 mEq/L. A 1 liter of fluid lost in urine can be replaced with ⅓ liter of I.V. fluid to achieve zero net sodium balance. As a result, theoretically, ⅔ liter of free water will be lost by the patient and no net loss of sodium will occur. Concentration of sodium in blood plasma will increase in proportion to the reduction of total body water. This example does not account for patient's drinking or for the water lost by evaporation.

One exemplary method includes the steps of administering the patient a diuretic to increase urine production, placing a urinary catheter in the patient, placing an infusion I.V. in the patient, collecting the urine from the patient, monitoring the volume of the collected urine, and automatically adjusting the rate of I.V. infusion based on the volume of the collected urine.

### BRIEF DESCRIPTION OF THE DRAWINGS

Other objects, features and advantages will occur to those skilled in the art from the following description of a preferred embodiment and the accompanying drawings, in which:

FIG. 1 is a schematic diagram of an example of a system for urine collection and infusion of hypertonic saline.

FIG. 2 is a schematic of the system electronics.

FIG. 3 is a chart of the software control algorithm for the system.

FIG. 4 is a flow chart of the operation of the system.

### DETAILED DESCRIPTION OF THE INVENTION

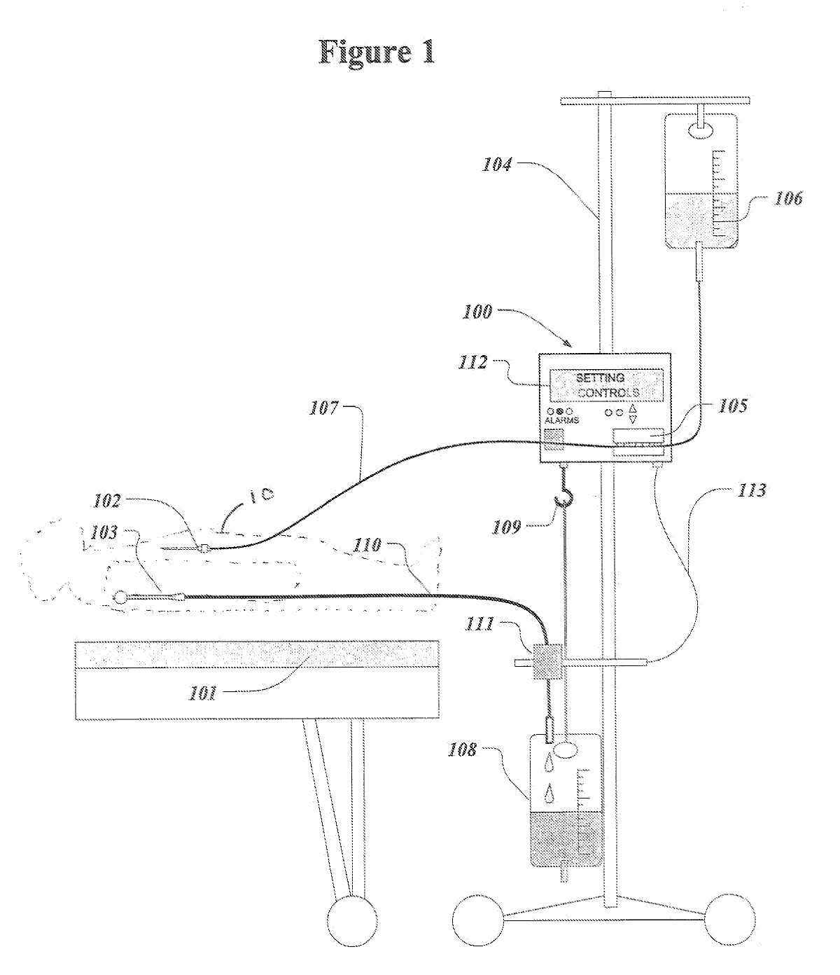

FIG. 1 schematically illustrates a controller console 100 comprising a programmable infusion pump, the controller electronics and the urine weighing mechanism. The patient 10 is placed on the hospital bed 101. The intravenous (I.V.) needle 102 and the urinary collection (Foley) catheter 103 are inserted into the patient to using standard methods. Console 100 is mounted on I.V. pole 104.

Console 100 typically includes an infusion device such as infusion pump 105 (e.g., a peristaltic pump) connected to source of infusion fluid 106 (e.g., hypertonic saline bag) by tubing 107. I.V. needle 102 is inserted in a vein of patient and is connected to infusion pump 105 via tubing 107.

Console 100 may include a weight scale such as an electronic load cell with a strain gage and other means to periodically detect the weight of the collected urine in chamber (i.e. urine collection bag or urine bag) 108. In the proposed embodiment, bag 108 with collected urine is hanging off the hook 109 connected to the load cell inside the console 100. The bag with fluid is suspended from the hook and a system of levers translate force to a scale such as strain gage. The strain gage converts force into an electronic signal that can be read controller. Suitable electronic devices for accurately measuring weight of a suspended bag with urine are available from Strain Measurement Devices, 130 Research Parkway, Meriden, Conn., 06450. These devices include electronics and mechanical components necessary to accurately measure and monitor weight of containers with medical fluids such as one or two-liter plastic bags of collected urine. For example, the overload proof single point load cell model S300 and the model S215 load cell from Strain Measurement Devices are particularly suited for scales, weighing bottles or bags in medical instrumentation applications. Options and various specifications and mounting configurations of these devices are available.

Other examples of gravimetric scales used to balance medical fluids using a controller controlling the rates of fluid flow from the pumps in response to the weight information can be found in U.S. Pat. Nos. 5,910,252; 4,132,644; 4,204,957; 4,923,598; and 4,728,433 incorporated herein by this reference. It is understood that there are many known ways in the art of engineering to measure weight and convert it into computer inputs. Regardless of the implementation, the purpose of the weight measurement is to detect the increasing weight of the collected urine in the bag 108 and to adjust the rate of infusion or hypertonic saline based on the rate of urine flow.

Urine collection bag 108 is connected by flexible tubing 110 to the Foley catheter 103 placed in the patient's urinary bladder to drain and collect urine in the standard fashion. Urine collected from the patient passes through the Sodium Concentration Sensor (Sodium Sensor) 111 on its way to the collection bag 108. The sodium sensor 111 is connected to the electronics (Not Shown) inside the Console 100 by the signal cable 113.

An example of a Sodium sensor can be an electrode manufactured by Microelectrodes, Inc. 40 Harvey Road Bedford, N.H. 03110, USA such as the MI-420 and MI-425 Na+ Ion microelectrodes. Sodium electrode can be used in combination with a separate Reference Electrode such as MI-409 if required. According to the manufacture, the MI-420 and Mi-425 are standardized using pure sodium chloride (NaCl) solutions and again in solutions containing possible interfering ions. Interference is significant when sodium concentration in urine is measured, since urine contains other conductive ions in addition to Na. The pure NaCl solutions can be used to determine probe function. In pure solutions, a 55 mv difference (approximate) will occur between each tenfold change in concentration. Standardization in solutions containing possible interfering ions is done in order to simulate the actual samples to be analyzed. For example, if your samples contain a known potassium background such as 100 millimoles KCl then your calibrating standards should also have this background.

The sensor 111 can be a urea sensor, instead of the sodium sensor. Urea is a suitable osmotic agent for the purpose of the invention. Many techniques for measurement of urea have been developed in the biomedical industry for analyzing biological fluids such as blood or urine so as to monitor renal function and for control of artificial dialysis. For example, U.S. Pat. No. 5,008,078, issued Apr. 16, 1991, inventors Yaginuma et al., describes an analysis element in which gaseous ammonia may be analyzed from liquid samples such as blood, urine, lymph and the like biological fluids. U.S. Pat. No. 5,858,186, issued Jan. 12, 1999, inventor Glass, describes a urea biosensor for hemodialysis monitoring which uses a solid state pH electrode coated with the enzyme urease and is based upon measuring pH change produced by the reaction products of enzyme-catalyzed hydrolysis of urea. There is also published research that demonstrates that concentration of both urea and sodium can be determined by spectral analysis. Modern technology of optical spectrometry can be adopted without excessive difficulty to allow rapid and reasonably priced determination of concentration of these molecules in urine. In “Online Measurement of Urea Concentration in Spent Dialysate during Hemodialysis” Jonathon T. Olesberg et. al. (Clinical Chemistry 50:1 175-181 (2004) Point-of-Care Testing) describe online optical measurements of urea using a Fourier-transform infrared spectrometer equipped with a flow-through cell in the effluent dialysate line during regular hemodialysis treatment of several patients.

Console 100 can be equipped with the user interface 112. The interface allows the user to set (dial in) the two main parameters of therapy. Display indicators on the console show the current status of therapy: the elapsed time and the total amount of urine made or the urine flow. The alarms notify the user of therapy events such as an empty fluid bag or a full collection bag as detected by the weight scale.

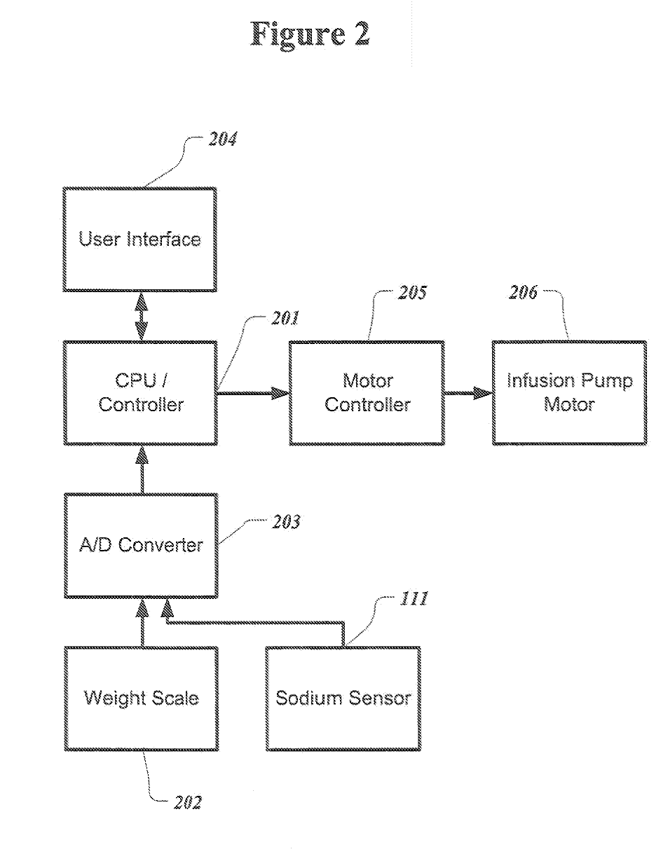

FIG. 2 is a block diagram of the electronic architecture of the controller console 100. CPU microprocessor 201 can be an integrated microcontroller that includes internal memory. Electronic signals from the weight scale 202 and the sodium sensor 111 are amplified and converted into digital information by the amplifier A/D converter 203. Resulting digital signals are periodically transmitted to the CPU 201 and stored in the CPU memory. These signals represent the volume of urine made by the patient and the concentration of sodium in the urine at the time when the measurement was made, for example every 100 milliseconds. User interface 204 can include dials, keys and displays commonly used in medical devices such as infusion pumps. User inputs such as commands to start and stop therapy or the information reflecting sodium concentration in the bag of the hypertonic saline is communicated to the CPU. CPU communicates to the user the information related to therapy such as the amount of urine made by patient, the amount of sodium excreted by patients and replaced by the I.V. infusion as well as alarms and other pertinent parameters. Inside the CPU 201 software algorithms combine the information received from sensors 202 and 111 and the user interface 204c input to generate electronic signal command to the motor controller 205 that can be a power amplifier or other device suitable to control the speed of the motor 206 of the infusion pump 105. The speed of the motor 206 is adjusted to achieve substantial balance of sodium: replace sodium lost in urine with the sodium infused by the pump.

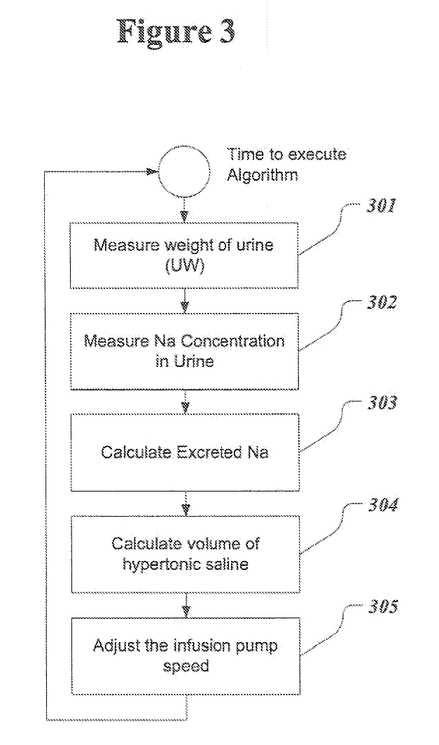

FIG. 3 is a flow chart that illustrates the elements of the software algorithm embedded in the CPU 201 of the controller Console 100. The algorithm maintains substantial balance of sodium in the patient's body while maximizing the excretion of water by the kidneys. Both volume (as approximated by weight) of urine 301 and concentration of sodium in urine 302 are measured, as described in other parts of the application, and combined 303 to calculate the amount of sodium excreted by the patient.

As indicated in TABLE I, total body water (TBW) content averages 60% of body weight in young men. About ⅔ of TBW is intracellular and ⅓ extracellular. About ¾ of the extracellular fluid (ECF) exists in the interstitial space and connective tissues surrounding cells, whereas about ¼ is intravascular.

TABLE I

| | | | Na | Na | |
|---|---|---|---|---|---|
| | | | Conc. | Conc. | Total Na |
| | Fraction | Liters | mEq/L | mg/L | grams |
| Total Body Weight BW | 100.0% | 70.0 | | | |
| Total Body Water TBW | 66.7% | 46.7 | | | 58.7 |
| Intracellular Fluid ICF | 44.4% | 31.1 | 12 | 276 | 8.6 |
| Extracellular Fluid ECF | 22.2% | 15.6 | 140 | 3,220 | 50.1 |
| Intravascular Volume | 5.6% | 3.9 | 140 | 3,220 | 12.5 |
| (plasma water) IVV | | | | | |
| Extravascular Water | 16.7% | 11.7 | 140 | 3,220 | 37.6 |
| EVS | | | | | |

There are significant differences in the ionic composition of intracellular fluid (ICF) and ECF. The major intracellular cation is potassium (K), with an average concentration of 140 mEq/L. The extracellular K concentration, though very important and tightly regulated, is much lower, at 3.5 to 5 mEq/L. The major extracellular cation is sodium (Na), with an average concentration of 140 mEq/L. Intracellular Na concentration is much lower at about 12 mEq/L and at 5 mEq/L. These differences are maintained by the Na+,K+-ATPase ion pump located in the cell membranes of virtually all cells. This energy-requiring pump couples the movement of Na out of the cell with the movement of K into the cell using energy stored in ATP.

The movement of water between the intracellular and extracellular compartments is largely controlled by each compartment's osmolality, because most cell membranes are highly permeable to water. Normally, the osmolality of the ECF (290 mOsm/kg water) is about equal to that of the ICF. Therefore, the plasma osmolality is a convenient and accurate guide to intracellular osmolality.

Normal blood Na should be in the range of 135-147 mEq/L. Abnormal blood plasma Na is termed hypernatremia when Serum Sodium over 147 mEq/L, and hyponatremia when Serum Sodium under 135 mEq/L. The proposed invention allows simple and safe control of blood Na for the physician.

To a physician, when adjustment of plasma Na is desired, it is important to change it slowly, rather than abruptly, to allow time for the redistribution of sodium in the total body water and to avoid the risk of arrhythmia or seizure from a transient and sudden high concentration of sodium in the blood stream entering the brain or the heart. It is also important to control the rate of change to prevent such problems as osmotic myelinolysis or central pontine myelinolysis. Simple ad-hoc calculations are commonly used in clinical practice to gradually control patient's blood sodium to a desired value. For example, for the infusion of normal saline (0.9%) with sodium concentration of 154 mEq/L (hypertonic saline can be substituted but is rarely used due to clinical concerns of patient safety), infused over the desired time at a desired rate, the resulting increase in plasma sodium can be calculated by the prescribing physician as follows:
Number of mEq/hr=Infusion pump rate (ml/hr)/1000×154 mEq/L A)
Serum Na increase per hour=mEq/hr/((Vd L/kg)×(Weight (kg))) where Vd (Volume of distribution)=0.6 L/kg Male or 0.5 L/kg Female B)
Total predicted serum sodium increase=(Serum Na+increase per hour)×Number of hours infused. C)

Exemplary calculation:

80 kg Male. Baseline serum sodium level: 132 meq/L, 0.9% NS infused at 150 ml/hr for 12 hours. Calculation of the projected serum sodium level after the completion of the 12 hour infusion.
(150 ml/hr) /1000×154 meq=23.1 meq/hr. A)
23.1 meq/hr /(0.6×80 kg)=0.48 meq/hr serum level increase. B)
Total predicted serum sodium increase=0.48 meq/hr×12 hrs=5.76 meq. C)
Predicted serum level=132+5.76=137.76 meq/L D)

A physician is cautioned that the actual serum sodium level obtained will depend on the patient's volume status, renal function, concomitant disease state(s), concurrent drug therapy and urine output. For example, if the patient was receiving loop diuretic and losing large amount of free water and sodium, the ad-hoc prediction will be incorrect and the resulting sodium in blood serum can be much higher or lower than expected. With the current technology this error is likely to be corrected no earlier than 12 hours later, when the therapy is completed and blood chemistry tests are done. Since blood samples are sent out to the lab, it may take up to 24 hours to find out how much the set rate of saline infusion was “off” or in error.

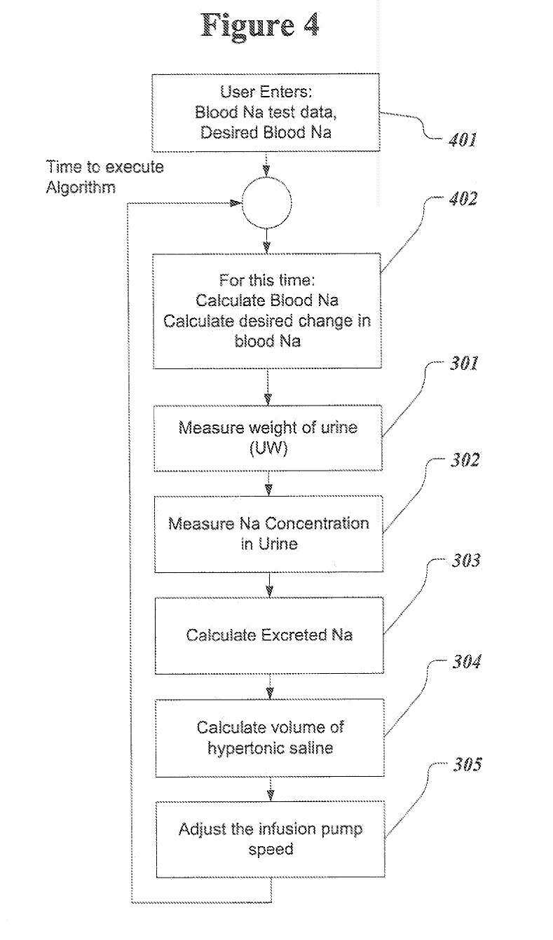

FIG. 4 illustrates the embedded algorithm of blood Na correction used by the controller. The details of the calculations are based on common equations of volume and mass balance (exemplified above) and need no detailed explanation for a person knowledgeable in performing such calculations manually. Embedding such calculations in software is well known in the field of control engineering. Unlike the calculations illustrated above, body water volume and total body water Na are not presumed to stay constant but automatically periodically corrected based on the excreted and infused Na and water. The infusion rate of the pump is corrected accordingly to achieve the goal of blood plasma Na concentration. At the beginning of the therapy, the user can enter a patient's weight, blood Na concentration (from lab tests), the desired blood Na at the end of therapy and the desired time to achieve that goal into the computer memory using the Console user interface 401. The System is then started. Every time the algorithm is executed by software (i.e. every 10 minutes), the “ins” and “outs” of water and sodium are recalculated 402 using most recent readings of sensors. In addition, the user may enter information such as oral intake of dietary sodium and water or the volume of water in additional injections. All this information is added up to calculate current blood plasma sodium concentration. This concentration is compared to the goal at that time. For example, if the therapy goal is to increase plasma Na from 130 to 140 mEq/L over 10 hours, at the time of five hours from the beginning of therapy the current goal can be 135 mEq/L. This current goal is compared with the calculated blood Na concentration that includes data from sensors an all up-to-date changes of body water and Na. After all the calculations are done, the infusion pump rate is adjusted and set until the next correction time period.

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

What is claimed is:

1. A patient therapy system comprising:
a source of a solution of a blood compatible osmotic solute,
an infusion pump and an intravenous (I.V.) set for controlled delivery of the solution to the patient,
at least one biofeedback sensor, and
a controller responsive to the biofeedback signals from the sensor configured to adjust the pump based on the output of the at least on biofeedback sensor.

2. The system of claim 1 in which the osmotic solute is hypertonic saline.

3. The system of claim 1 in which the osmotic solute is urea.

4. The system of claim 1 in which the at least one biosensor includes a urine volume sensor and a sodium concentration sensor.

5. A method of controlling infusion of an osmotic solute comprising:
infusing the osmotic solute into a patient at a controlled infusion rate;
sensing a condition of the patient, wherein the condition is influenced by the infused osmotic solute, and
adjusting the infusion rated based on the sensed condition.

6. A method as in claim 5 wherein the osmotic agent is hypertonic saline.

7. A method as in claim 5 wherein the osmotic agent is urea.

8. A method as in claim 5 further comprising sensing the sensed condition using at least one biosensor.

9. A method as in claim 8 wherein the at least one biosensor includes a urine volume sensor and a sodium concentration sensor and the sensed condition includes at least one of urine volume of the patient and sodium concentration in the urine.

10. A method of increasing a urine production of a patient comprising:
infusing an osmotic agent into a blood of the patient at a controlled infusion rate;
measuring urine output of the patient;
measuring a concentration of the osmotic agent in the urine output by the patient, and
automatically adjusting the controlled infusion rate based on the measured concentration of the osmotic agent in the urine.

11. A method as in claim 10 further comprising automatically adjusting the controlled infusion rate based on a volume of the urine output.

12. A method as in claim 10 wherein the volume of the urine output is measured over a predetermined period of time.

13. A method as in claim 10 wherein the osmotic agent is hypertonic saline.

14. A method as in claim 10 wherein the osmotic agent is urea.
